(19) United States
(12) Patent Application Publication
(10) Pub. No.: US 2004/0267309 A1
(43) Pub. Date: Dec. 30, 2004

(54) Device for sutureless wound closure

(76) Inventor: Dennis D. Garvin, Roanoke, VA (US)

Correspondence Address: Intellectual Property Department, DEWITT ROSS & STEVENS S.C., US Bank Building, 800 Excelsior Drive Suite 401, Madison, WI, 53717-1914, US

(21) Appl. No.: 10/607,650

(22) Filed: Jun. 27, 2003

(51) Int. Cl. A61D 1/00; A61B 17/08

(52) U.S. Cl. 606/217; 606/216; 606/213

(57) ABSTRACT

The invention discloses a device and technique for a sutureless wound closure, which limits the risks of rupture and scarring. The device comprises a two-part strap having short hooks or barbs on the ventral surface. The barbs are curved and flexible such that, when urged in one direction, the barbs do not engage while when urged in the opposite direction, the barbs are pushed downward and engage the tissue of the wound. By deploying one strap on each side of the wound, fastening the two straps in the middle of the wound and tightening the ends, the wound is closed. The tension required to close the wound is distributed along the length of the straps, thus resulting in a cleaner, safer closure.

### FIELD OF THE INVENTION

The present invention relates to a surgical fastener and technique for its use. Specifically, the invention is directed to a device to close wounds without the use of sutures.

### DESCRIPTION OF THE PRIOR ART

Classical techniques to close wounds and incisions use sutures, basically using a needle and thread to sew the wound closed. While this technique acts to stitch the respective sides of the lesion together, it has several drawbacks. First, the tension required to pull the sides together is localized at the point of the stitch. This results in a tendency of the skin to tear around the stitch. Second, the skin may pouch out or sacculate between the stitches, greatly increasing the susceptibility of the wound to infection. Third, because the two sides of the wound are not evenly juxtaposed, scarring along the path of the sutures is increased. In addition, the placement of sutures requires deployment of needle and filament and afterward the tying off of the ends of the filament. This process is time consuming and requires workspace allowing dexterous manipulation.

Prior devices and techniques have been developed in an attempt to resolve these problems. These techniques range from superficial wound closure techniques to internal repair techniques. For example, U.S. Pat. No. 3,971,384 to Hasson describes a surgical closure device designed to bring the two edges of a wound or incision together. A piece of surgical tape is secured on each side of the wound. One piece of tape has an anchor for a tie strip secured to it while the other piece of tape has a slide secured to it. The tie strip has ratchet teeth on its dorsal surface such that the strip is inserted through the anchor end, across the wound and into the ratchet. The tape is then tightened and locked with the ratchet, bringing the two sides of the wound together. U.S. Pat. No. 4,924,866 to Yoon describes a device for closing wounds comprising two arms connected by a hinged joint. The arms have a single pair of “skin engaging members” on the ventral surface such that when the device is placed. over the wound, the members enter the skin, pulling the wound together underneath the joint.

While the devices described by Hasson and Yoon are directed to sutureless methods of wound closure, they suffer from certain defects. In particular, Hasson is limited to superficial applications where the tape can stick and, further, by the strength and size of the tape. The described device can receive no more force from the opposing sides of the wound than the tape can hold. In addition, the size of the device is limited by the size of the tape. The device of Yoon is similarly handicapped. First, the device is limited in its pliability by the structure of the arms. Second, the device is limited in its wound closure ability due to the limited number of “skin engaging members” in relatively close proximity to the wound. Third, because there is no ratcheting element, the sides of the wound must first be properly juxtaposed and aligned before its insertion as there is no second chance for its deployment.

Other devices have been described for internal tissue repair or reconstruction. They include U.S. Pat. No. 6,241,747 to Ruff, which describes a barbed tissue connector for closing tissue wounds. The connector comprises an elongated shaft with pointed ends and a multitude of circumferentially placed barb-like points along the length of the shaft. The shaft has a midline with the barbs on either side pointing away from the midline and toward the respective ends. In use, the tip of one end is inserted into one side of the wound. The wound is spread apart and the other end of the device inserted. After each end is inserted into the wound, the tissue is pressed together with the fingers to fully engage the barbs and bring the sides of the lesion into express contact. Because the device of Ruff has no dorsal or ventral surface, it must be placed deep enough in the lesion such that the tip of the circumferential barbs remains within the skin. Such means of insertion adds to the trauma already experienced by the tissue.

U.S. Patent Application Publication 0058966 to Tormala et al. describes a surgical fastener or implant for repairing tissue wounds, particularly torn menisci in the knee. The invention comprises a shaft with an arrow-like point on one end and a blunted barb on the other end. The barbs on both ends of the shaft are directed such that they point toward the ends of the shaft, thereby facilitating insertion and discouraging its removal. The barbed end of the device is passed through both ends of the cartilage where the ends are locked onto the shaft by the inwardly pointed ridges of the blunt end. The device described by Tormala requires use with a structure dense enough to have the device embedded within it and is thereby limited in its use.

### SUMMARY OF THE INVENTION

The present invention is directed to a sutureless wound closure device that eliminates the pocketing and rupture associated with traditional sutures. Further, the device allows the tension, of pulling the opposing sides of the wound together, to be spread over a large area of the adjacent tissue. Also, the device is easy to use and does not further increase the trauma already experienced by the underlying tissue.

In a preferred embodiment, the invention comprises a wound closure device for connecting tissue comprising a first and second strap, each strap including a ventral barbed surface. The straps are adjustably connectable to one another, whereby the straps form a wound closure.

In another preferred embodiment, the invention comprises a wound closure device for connecting tissue having a first flexible strap with a proximal end, distal end, ventral surface and dorsal surface. Also included is a second flexible strap also having a proximal end, distal end, ventral surface and dorsal surface. The proximal end of the second strap terminates in a connector designed and configured to adjustably connect to the proximal end of the first strap. Further, the first strap and the second strap have at least one barb on the ventral surface for engaging the tissue. By inserting each strap on either side of the wound, connecting the straps and adjusting them so as to bring the sides of the wound together, the straps form a wound closure.

In yet another version of the invention, the device comprises a first and second strap. Each strap includes a ventral surface having at least one barb, and a proximal and distal end. The distal end of each strap is placed in tissue surrounding the wound, and the proximal end of each first strap is designed and configured to be adjustably connected to the proximal end of each second strap. By connecting and adjusting the straps, the device forms a wound closure.

The advantages of the invention are manifold. First, from a clinical standpoint, the invention helps to limit rupture of the wound. Second, from a cosmetic standpoint, the invention greatly limits scarring by reinforcing the subcutaneous fascia and eliminating sutures. Third, due to the above two advantages, the invention greatly reduces infection. Fourth, the invention is less painful and the patient heals faster than traditional wound closure methods because staples or sutures, piercing through the underlying muscle, are not required.

The invention can also be used in most settings and locales from acute and field conditions to chronic conditions treated in care facilities. For example, the invention can be used for closure of small laparoscopy ports, which is difficult, particularly in obese patients. In these conditions, standard suturing through a small skin incision is very difficult and takes significant time or requires a larger skin incision to be made. Thus, the invention can make more demanding procedures easier and allow time-consuming procedures to be performed in more urgent situations.

The invention also allows greater blood flow to the healing tissue. When a conventional stitch is used under high tensions, it results in blood being cut off to the tissue encircled by the loop of the stitch. In contrast, by using the present invention, this problem is alleviated. Allowing greater blood flow to the incision reduces scarring and results in much better results, particularly with cosmetic surgery.

Further, the straps can be modified. Such modifications can allow the use of the invention in tightening waistlines, which have been stretched by injury, surgery or childbirth. The straps can also be applied to the top of a hernia repair to reduce risk of recurrence or adapted to facilitate a sternotomy closure, which would stabilize the chest and reduce discomfort after open-heart surgery.

The objects and advantages of the invention will appear more fully from the following detailed description of the preferred embodiment of the invention made in conjunction with the accompanying drawings.

### BRIEF DESCRIPTION OF THE DRAWINGS

FIG. 1 is a perspective view of the first strap being inserted into the connector of the second strap and adjusted, thereby closing the wound.

FIG. 2a is a partial side elevation view of the first strap of the device at the proximal end.

FIG. 2b is a ventral elevation view of the strap of FIG. 2a.

FIG. 2c is a partial side elevation view of the second strap of the device at the proximal end.

FIG. 2d is a ventral elevation view of the strap of FIG. 2c.

FIG. 3a is a side elevation illustrating the flexion of the barbs of the closure device while stored in a trochar.

FIG. 3b is a perspective view of the device being deployed out of the trochar, shown in a cut-away view, with the barbs of the device being unflexed.

### DETAILED DESCRIPTION OF THE INVENTION

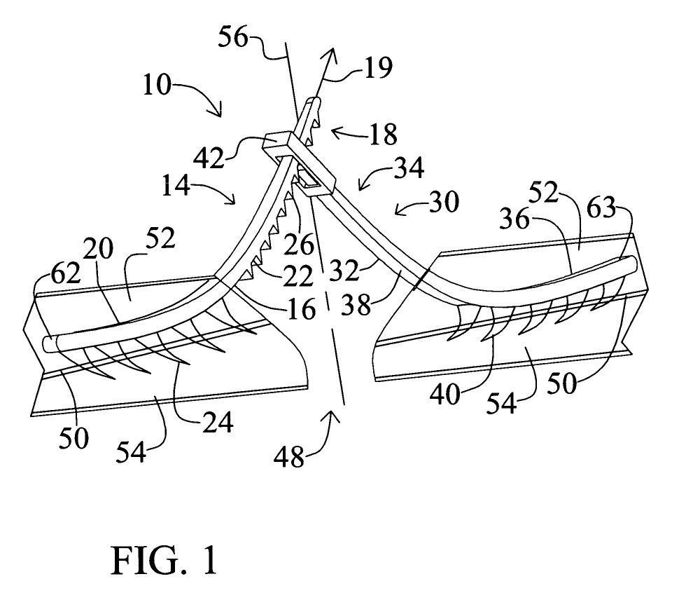

Referring now to FIG. 1, there is illustrated a perspective view of a preferred embodiment of the disclosed invention 10. As illustrated, the invention 10 includes a first strap 14 and a second strap 30. The first strap 14 and the second strap 30 include distal ends 62 and 63 and proximal ends 18 and 34, respectively. The straps 14 and 30 are planar and made of a pliable material having dorsal surfaces 20 and 36 and a ventral surface 22 and 38. The dorsal surface 20 and 36 of the straps 14 and 30 are smooth while the ventral surfaces 22 and 38 include a plurality of small barbs 24 and 40 which project downward from the ventral surface 22 and 38 and curve toward the proximal end 18 and 34. In one preferred embodiment, the straps 14 and 30 have a length of about 4 cm and a width of about 0.5 cm. In other embodiments, the straps 14 and 30 may be larger or smaller to accommodate a wound, illustrated at 48. Although the shape of the straps 14, 30 may include planar sides as illustrated in FIG. 1, it is within the scope of the present invention for each strap to have a rounded configuration.

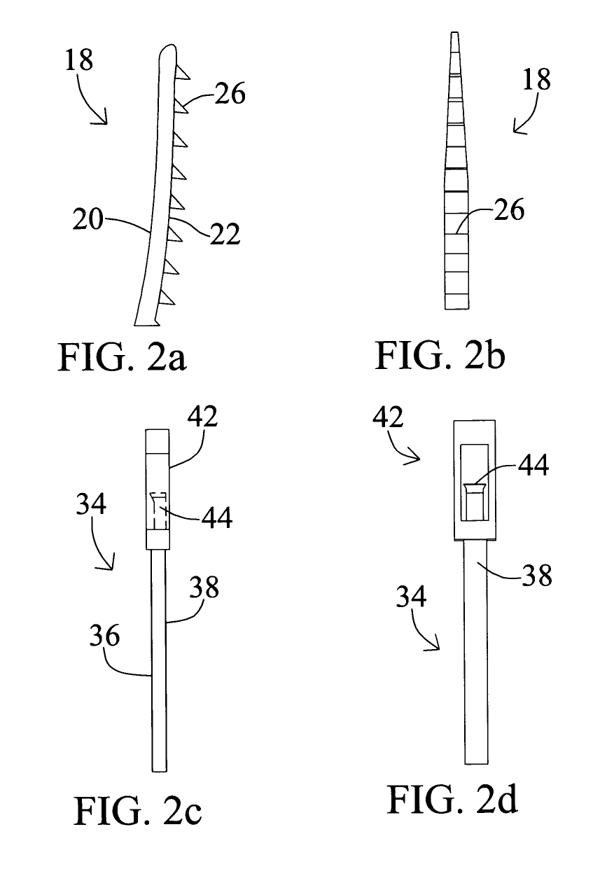

As shown in FIG. 1 and FIGS. 2a and b, the proximal end 18 of the first strap 14 is tapered along its planar sides, and the ventral surface 22 is composed of a plurality of small teeth or ratchets 26. The ratcheted surface may comprise about between 1 and 20 mm of the proximal end 18 of the first strap 14. Between the end of the ratcheted surface and the beginning of the barbs 24, there is a gap space 16 on the ventral surface 22, which is smooth and has no protuberances.

FIG. 1 and FIG. 2c and d illustrate a preferred version of the second strap 30. In this version the proximal end 34 of the second strap 30 terminates in a buckle 42. However, in other versions, the proximal end 34 may terminate in any other form of connector, which may be capable of adjustably connecting the two straps such as VELCRO, adhesives or clips. Between the buckle 42 and the most proximal of the barbs 40 is a gap space 32 marked by a smooth region of the ventral surface.

As illustrated in FIG. 1 the invention 10 is deployed in a lesion or wound 48. In use, the first strap 14 and the second strap 30 of the device 10 are placed on either side of the wound 48 in an orientation perpendicular to the axis of the wound 56. In a preferred embodiment, the straps 14 and 30 are inserted underneath the epidermis directly above the fascia 50 of the surrounding tissue. The distal end 62 and 63 of each strap 14 and 30 are directed away from the lesion while the proximal ends 18 and 34 of the straps 14 and 30 are situated at about the midline 56 of the wound 48. The straps 14 and 30 are displaced on either side of the wound 48 such that the gap space 16 and 32 of each strap 14 and 30 is generally behind the edge of the wound 48. The barbs 24 and 40 are then gently embedded in the underlying tissue, which in a preferred version of the invention is the fascia 50, so that the straps 14 and 30 engage the tissue.

FIG. 1 shows the barbs 24 and 40 of the straps 14 and 30 engaged in the fascia 50 with the barbs 24 and 40 pointed toward the midline 56 of the wound 48. The two straps 14 and 30 are connected at their proximal ends 18 and 34 and adjusted to a desired tightness. Illustrated is one preferred version of the invention showing the straps 14 and 30 that are tightened by pulling the proximal end 18 of the first strap 14 through the buckle 42 along the path designated by the arrow 19. The buckle 42 is then tightened by urging it distally on the ratchets 26 of the first strap 14. Tightening or putting tension on the proximal ends 18 and 34 of the straps 14 and 30 pulls the underlying fascia 50 of the wound together, allowing a smooth joining of the tissue surrounding the wound 48.

When the wound is large or the tissue is delicate, multiple straps 14 and 30 may be needed. When multiple straps are used, the straps 14 and 30 are deployed on either side of the wound 48. When all the straps 14 and 30 are deployed along the length of the lesion, the tapered, proximal end 18 of each first strap 14 is inserted into the buckle 42 of the respective second strap 30 until the locking tongue 44 engages the ratchets 26 of the first strap 14. In a particularly preferred version, the locking action of the first proximal end 18 in the buckle 42 is like that of a nylon tie such that once the tongue 44, illustrated in FIG. 2d, is engaged with the ratchets 26, the tension on the straps 14 and 30 can be increased by pulling the proximal end 18 through the buckle 42 in the direction of the arrow 19. The process of pulling the proximal end 18 of the first strap 14 through the buckle 42 of the second strap 30 of each of the respective first 14 and second 30 pair of straps, allows the opposing sides of the wound 48 to be brought close enough to begin tightening the individual straps in a sequential fashion until the opposing sides of the wound 48 are brought together.

Because the barbs 24 and 40 continue into the tissue surrounding the wound along the length of the straps 14 and 30, the tension loaded on the straps 14 and 30 is transferred to the underlying tissue assuring a smooth juxtaposition of the opposing sides of the wound 48. It will be appreciated that depending on the size of the wound 48, more or less straps 14 and 30 may be needed. For example, a very large wound 48 will require a large number of straps 14 and 30 while a small wound 48 will require one or a few straps 14 and 30. Similarly, very delicate or visible tissue may require many small straps 14 and 30 while tough or concealed tissues may require fewer large straps 14 and 30.

As shown in FIG. 1, the barbs 24 and 40 are conical in shape, ending at a point. The barbs 24 and 40 are designed to be pliable yet have stiffness such that they can pierce tissues ranging from muscle to skin to fat. Such barbs can be made from nylon, plastics and resorbable polymers such as polyglycolic acid and poly-L lactic acid, for instance. The barbs 24 and 40 may have a slightly different shape, depending on the particular tissue to be used in. For example, straps to be used in adipose tissue 52 may have barbs 24 and 40 that are longer and broader because the tissue is soft while barbs 24 and 40 to be used in muscle 54 or connective tissue, such as the fascia 50, may be shorter and narrower because those tissues are tough, and the barbs 24 and 40 do not need to project far into the tissue to embed. Nevertheless, the barbs 24 and 40 should generally be about between 2.5 mm long and 4.0 mm long and have a circumference around the base of about 2-3 mm. Further, while one exemplary version of the invention has only one barb 24 and 40 per row along the horizontal axis of the straps 14 and 30, in other exemplary versions, there may be several barbs 24 and 40 per row arranged along the ventral surface 22 and 38 of the straps 14 and 30.

FIGS. 2a-d illustrate a particularly preferred version of the proximal ends 18 and 34 of the first and second straps 14 and 30. FIGS. 2a and 2b show a side and ventral elevation, respectively, of the proximal end 18 of the first strap 14. As shown, the first strap 14 is tapered toward the proximal end 18 and has a series of small ratchet-like protrusions 26 on its ventral surface 22 while the dorsal surface 20 is flat. FIGS. 2c and 2d show a side and ventral elevation, respectively, of the proximal end 34 of the second strap 30. FIG. 2c shows the dorsal surface 36 and the ventral surface 38 with the buckle 42 terminating the proximal end 34 of the second strap 30. FIG. 2d illustrates the ventral surface 38 of the proximal end 34 of the second strap 30 with the buckle 42 terminating the proximal end and a locking tongue 44 situated within the buckle 42.

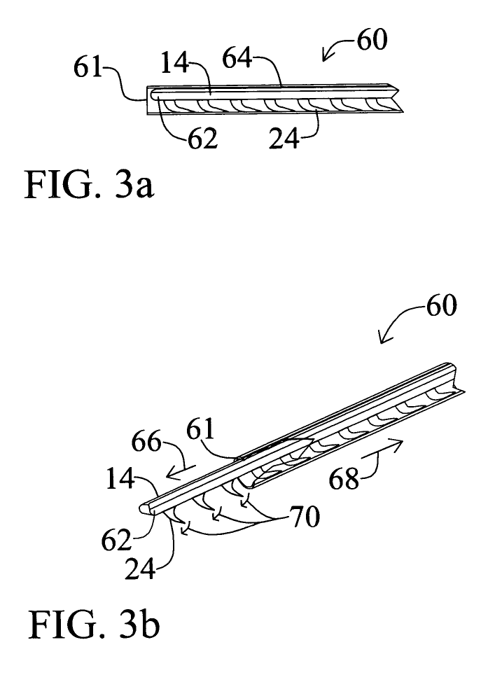

Methods to aid in deployment of the invention 10 are also included. For example, FIG. 3a illustrates the strap 14 inside a trochar 60. For the purposes of the drawing, only the second strap 14 is illustrated. However, both the first strap 14 and the second strap 30 are deployed with the trochar 60 and will have similar dimensions with comparable distal ends 62 and 63 as shown in FIG. 1. The trochar 60 is a cylindrical tube having walls 64 fabricated from a material stiff enough to use as an applicator. In particularly preferred versions, the trochar 60 may be made of, plastic or metal and will have a diameter slightly greater than the width of the straps 14 and 30 it is used to deploy. While in the trochar 60, the barbs 24 and 40 are flexed upward due to the slightly greater length of the barbs compared to the height of the trochar 60. The trochar 60 may serve both to store the straps 14 and 30 in and as an applicator for the straps 14 and 30. In situations where the trochar 60 is disposable, the straps would come stored in the trochar. In situations where the trochar 60 is reloadable, the straps 14 and 30 and the trochar 60 may be stored separately.

FIG. 3b illustrates the movement of the barbs 24 and 40 downward, shown by the arrows 70, as the straps 14 and 30 are slid out of the opening 61 of the trochar 60. In a preferred version of the invention, the straps 14 and 30 are deployed by sliding the end of the trochar 60 containing the distal end 62 of the strap 14 and 30 between the fascia 50 and the overlying adipose layer 52 of the tissue of the wound 48 (shown in FIG. 1). The distal ends 62 or 63 of the straps 14 or 30 are then urged out of the trochar 60 from the proximal end 18 or 34 in the direction of the arrow 66, deploying the most distal barbs 24 and 40 into the fascia 50. The trochar 60 is then pulled off the remainder of the strap 14 or 30, in the direction of the arrow 68, embedding the barbs 24 and 40 in the underlying fascia 50. This process is repeated for the opposing strap such that the proximal end 18 of the first strap 14 and the proximal end 34 of the second strap 30 abut each other at about the midline 56 of the wound 48. The proximal end 18 of the first strap 14 is then inserted in the buckle 42 of the second strap and tightened by pulling the first strap 14 in the direction of the arrow 19 as previously described.

It is a further facet of the invention 10 that while, in a preferred embodiment, the straps 14 and 30 are deployed subcutaneously in the fascia 50, as shown in FIG. 1., the straps may also be deployed on the surface of the skin with the barbs 24 and 40 engaged with the epidermis. The straps 14 and 30 may also be used internally in most situations where conventional stitches are used such as during exploratory surgery or resections, and with most tissues, including connective tissues, such as tendons, cartilage, ligaments and adipose tissue.

It is yet another facet of the invention 10 that the straps 14 and 30 are made out of any hypoallergenic material and may be resorbable or permanent. In some instances, the straps 14 and 30 may not be resorbable and will remain engaged in the lesion or wound 48, such as permanent sutures are, or until the care provider elicits their removal. In other versions of the invention, the straps may be made of resorbable materials such as those described in U.S. Pat. No. 4,968,317 to Tormala et al. or U.S. Pat. No. 4,898,186 to Ikada et al., both hereby incorporated by reference for their description of such materials.

It is understood that the invention is not confined to the particular construction and arrangement of parts herein illustrated and described but embraces such modified forms thereof as come within the scope of the following claims.

What is claimed is:

1. A wound closure device for connecting tissue comprising: first and second straps, each including a ventral surface having at least one barb, the straps being adjustably connectable to one another, whereby the straps form a wound closure.

2. The device of claim 1, wherein the barbs of the first strap and the second strap are placed in the fascia of the wound.

3. The device of claim 1, wherein the first strap and second strap are made of resorbable material.

4. The device of claim 1, wherein the device is placed in the wound by the use of a trochar.

5. The device of claim 1, wherein the ventral surface of each strap includes a plurality of barbs.

6. A wound closure device for connecting tissue comprising:
a. a first flexible strap having a proximal end and a distal end, and a ventral surface and a dorsal surface; and
b. a second flexible strap having a proximal end and a distal end, and a ventral surface and a dorsal surface, wherein the proximal end terminates in a connector designed and configured to adjustably connect to the proximal end of the first strap,
wherein the first strap and the second strap have at least one barb on the ventral surface for engaging the tissue;
whereby the straps form a wound closure.

7. The device of claim 6, wherein the first strap and the second strap are placed in the fascia of the wound.

8. The device of claim 6, wherein the device is made of resorbable material.

9. The device of claim 6, wherein a plurality of barbs is present on the ventral surface of the first and second straps.

10. A wound closure device for connecting tissue comprising:
first and second straps, each strap including
a. a ventral surface having at least one barb; and
b. a proximal and distal end;
wherein
a. the distal end of each strap is placed in tissue surrounding the wound, and
b. the proximal end of each first strap is designed and configured to be adjustably connected to the proximal end of each second strap,
whereby the straps form a wound closure.

11. The device of claim 10, wherein the barbs of the first strap and the second strap are placed in the fascia of the wound.

12. The device of claim 10, wherein the device is made of resorbable material.

13. The device of claim 10, wherein the device is placed in the wound by the use of a trochar.

14. The device of claim 10, wherein the first and second strap each has a plurality of barbs on its ventral surface.

15. The device of claim 10, wherein the proximal end of the first strap comprises a male end and the proximal end the second strap comprises a female end.

16. The device of claim 15, wherein the female end comprises a buckle.
